(19) United States
(12) Patent Application Publication
(10) Pub. No.: US 2025/0257323 A1
(43) Pub. Date: Aug. 14, 2025

(54) ACTIVATION-INDUCED TISSUE-EFFECTOR CELLS SUITABLE FOR CELL THERAPY AND EXTRACELLUAR VESICLES DERIVED THEREFROM

(71) Applicants: Capricor, Inc., Beverly Hills, CA (US); Cedars-Sinai Medical Center, Los Angeles, CA (US)

(72) Inventors: Ahmed G. Ibrahim, Los Angeles, CA (US); Luis Rodriguez-Borlado, Manhattan Beach, CA (US); Chang Li, Harbor City, CA (US); Jennifer J. Moseley, Northborough, MA (US); Eduardo Marbán, Santa Monica, CA (US)

(21) Appl. No.: 19/061,766

(22) Filed: Feb. 24, 2025

Related U.S. Application Data
(63) Continuation of application No. 16/965,835, filed on Jul. 29, 2020.
(60) Provisional application No. 62/623,927, filed on Jan. 30, 2018.

(51) Int. Cl. C12N 5/077; A61K 35/34; A61K 48/00; A61P 9/10; A61P 29/00

(52) U.S. Cl. CPC C12N 5/0657; A61K 35/34; A61P 9/10; A61P 29/00; A61K 48/00; C12N 2501/415; C12N 2501/60; C12N 2501/606; C12N 2510/04; C12N 2533/52

(57) ABSTRACT

The present invention provides a method of inducing activation of a non-potent or insufficiently potent cell to convert the cell into a tissue-effector cell, thereby producing an activation-induced tissue-effector cell suitable for use in cell therapy—e.g., an activated specialized tissue-effector cell (ASTEC) suitable for cell therapy for a particular tissue type. The present invention further provides activation-induced tissue-effector cells produced thereby, as well as extracellular vesicles, e.g. exosomes, derived therefrom (e.g., ASTEX). The present invention further provides a method of improving the efficacy of a cell therapy by converting non-potent or insufficiently potent cells into activation-induced tissue-effector cells having increased potency suitable for cell therapy. The present invention further provides a method for treating a disease or condition amenable to cell therapy in a subject in need thereof, the method comprising administering a therapeutically effective amount of activation-induced tissue-effector cells or extracellular vesicles derived therefrom.

### BACKGROUND OF THE INVENTION

WO/2006/052925 (entitled “cardiac stem cells”) and US 2012/0315252 (entitled “methods of reducing teratoma formation during allogeneic stem cell therapy”) describe cardiosphere-derived cells (CDCs), their derivation from cardiospheres, and their therapeutic utility for increasing the function of a damaged or diseased heart of a mammal. WO/2005/012510 (entitled “method for the isolation and expansion of cardiac stem cells from biopsy”) describes cardiospheres, their derivation from cardiac tissue biopsy samples, and their therapeutic utility in cell transplantation and functional repair of the myocardium. WO/2014/028493 (entitled “exosomes and micro-ribonucleic acids for tissue regeneration”) describes exosomes derived from CDCs and their therapeutic utility for the repair or regeneration of damaged or diseased cardiac tissue. WO/2014/066545 (entitled “therapeutic cells depleted of specific subpopulations of cells for use in tissue repair or regeneration”) describes that CDCs from which a subpopulation of cells expressing CD90 have been removed, i.e., CD90-depleted CDCs, have increased potency to treat diseased or damaged cardiac tissue.

However, it remains unknown as to what drives increased cell potency in CD90-depleted CDCs, and thus the current state of art is limited to using CD90-depleted CDCs in the hope of increasing cell potency.

### SUMMARY OF THE INVENTION

The present invention is based on the surprising discovery by the present inventors that exogenously increasing the level of β-catenin in certain non-potent or insufficiently potent cells induces those cells to be activated and be converted into tissue-effector cells, thereby producing activation-induced tissue-effector cells suitable for use in cell therapy. The present inventors further discovered that certain non-potent or insufficiently potent cells require exogenously increasing the levels of one or more additional transcription factors of interest, e.g., GATA4, in addition to exogenously increasing the level of 0-catenin, to become activated specialized tissue-effector cells (ASTECs) suitable for use in cell therapy for a particular tissue lineage, e.g., to treat a disease or disorder of the cardiac tissue. The present invention as described herein thus provides an innovative cell therapy modality wherein ASTECs, as well as extracellular vesicles derived from ASTECs (ASTEX), can be prepared and configured to treat a particular disease or disorder of a particular tissue or organ in a patient.

A first aspect of the present invention provides a method of increasing the therapeutic potency of a cell or a cell population, the method comprising the step of contacting the cell or the cell population with an exogenous agent to increase or boost the cellular level of the transcription factor β-catenin thereof, thereby producing an activation-induced tissue-effector cell or a particular population of activation-induced tissue-effector cells. In a preferred embodiment, the cell is a mammalian cell, and more preferably a human cell, and the cell population is a mammalian cell population, and more preferably a human cell population. Non-limiting examples of the cell population include cardiospheres, CDCs, explant-derived cells (EDCs) as described in, e.g., US 2012/0315252, newt A1 cells, fibroblasts such as normal human dermal fibroblasts (NHDFs), other stromal cells such as epithelial cells, endothelial cells, smooth muscle cells, keratinocytes, chondrocytes, neurons, glial cells, pericytes, and muscle satellite cells. Non-limiting examples of the exogenous agent for increasing the level of β-catenin of the cell or the cell population include inhibitors of glycogen synthase kinase-30 (GSK-30) and activators of the Wnt signaling pathway, such as 6-bromoindirubin-3′-oxime (BIO), Wnt3a, and CHIR (e.g., CHIR99021). In several embodiments, the method further comprises the step of contacting the cell or the cell population with one or more additional exogenous agents to increase the level of one or more additional transcription factors, either before or after the step of contacting the cell or the cell population with an exogenous agent to increase the level of β-catenin thereof, thereby producing an ASTEC or a particular population of ASTECs having therapeutic effects with respect to, e.g., T-cell polarity, neural inhibition, tissue regeneration, anti-cancer, anti-aging, axonal remyelination, macrophage polarity, angiogenesis, and cardiac pacing. Non-limiting examples of said additional transcription factors include Tbet, GATA3, GATA4, Ascl1, Ptf1a, Pax6, MCPIP, PPARy, KLF4, Sox10, Hey 1/2, mesoderm-specific transcript (mest), and Tbx18. For instance, the mammalian cell is first contacted with an exogenous agent to increase the level of β-catenin of the cell, and then with another exogenous agent to increase the level of GATA4 of the cell; alternatively, the mammalian cell is first contacted with an exogenous agent to increase the level of GATA4 of the cell, and then with another exogenous agent to increase the level of β-catenin of the cell, thereby producing an ASTEC suitable for treating a disease or disorder of the cardiac tissue. In several embodiments, the method further comprises the step of selecting the resulting activation-induced tissue-effector cells for use in cell therapy. In several embodiments, the cell or the cell population is non-potent, borderline-potent, marginally-potent, or insufficiently potent, which attains sufficient potency upon conversion to an activation-induced tissue-effector cell or a particular population of activation-induced tissue-effector cells. In several embodiments, the cell or the cell population is already sufficiently potent, which attains every greater potency upon conversion to an activation-induced tissue-effector cell or a particular population of activation-induced tissue-effector cells. In several embodiments, the cell or the cell population is an immortalized cell or an immortalized cell population. For example, immortalized CDCs are produced by overexpressing simian virus 40 large and small T antigens (SV40 T+t), or c-Myc, in a culture of CDCs, and selecting a CDC culture that can continue to double for, e.g., at least 10 times, or preferably at least 15 times. In several embodiments, the cell or the cell population is plated on a fibronectin-coated culture vessel.

A second aspect of the present invention provides an activation-induced tissue-effector cell or a particular population of activation-induced tissue-effector cells, e.g., immortalized CDCs, or NHDFs, with increased levels of β-catenin and GATA4, which are suitable for treating cardiac disease or disorder, prepared by the method according to the first aspect of the present invention.

A third aspect of the present invention provides extracellular vesicles derived from activation-induced tissue-effector cells, wherein activation-induced tissue-effector cells are prepared by the method according to the first or the second aspect of the present invention. For example, immortalized CDCs, or NHDFs, are converted to ASTECs by increasing their levels of β-catenin and GATA4 in the manner according to the first or the second aspect of the present invention, from which extracellular vesicles are harvested. In some embodiments, extracellular vesicles are harvested under serum-free and/or hypoxic conditions. Non-limiting examples of extracellular vesicles derived from activation-induced tissue-effector cells mainly include exosomes and microvesicles, and may also include membrane particles, membrane vesicles, exosome-like vesicles, ectosomes, ectosome-like vesicles, exovesicles, epididimosomes, argosomes, promininosomes prostasomes, dexosomes, texosomes, archeosomes, and oncosomes.

A fourth aspect of the present invention provides a pharmaceutical composition, formulation, or preparation comprising a therapeutically effective amount of activation-induced tissue-effector cells prepared by the method according to the first or the second aspect of the present invention and/or extracellular vesicles derived from activation-induced tissue-effector cells according to the third aspect of the present invention.

A fifth aspect of the present invention provides a method of treating a disease or disorder associated with a particular tissue or organ in a subject in need thereof, the method comprising administering to the subject a therapeutically effective amount of activation-induced tissue-effector cells prepared by the method according to the first aspect of the present invention, extracellular vesicles derived from activation-induced tissue-effector cells according to the third aspect of the present invention, and/or the pharmaceutical composition, formulation, or preparation according to the fourth aspect of the present invention. Non-limiting examples of said disease or condition include autoimmune disease, neuropathies, aging, spinal cord injury, vascular disease, neuromuscular disorders, cancer, fibrotic diseases, cardiac arrhythmias, heart failure, myocardial infarction, and primary and secondary malignancies. In several embodiments, said administration is via subcutaneous injection, transcutaneous injection, intradermal injection, topical administration (e.g., in the form of eye drops), intramyocardial injection, injection into lymphoid tissue, injection into the lymphatic system, systemic administration (e.g., oral, intravenous, intraparenteral), or the like.

As a non-limiting example, what is meant by “potent” or “sufficiently potent” cells according to the present invention is that such cells are capable of improving a particular disease state by an appreciable degree as measured by a mouse model of acute myocardial infarction. For instance, administration of “potent” ASTECs in the heart of an infarcted mouse would increase the left ventricular ejection fraction by at least 2% (ΔEF≥2%), and more preferably by at least 4% (ΔEF≥4% improvement at day 21 compared to day 1). See, e.g., Smith et al., Regenerative potential of cardiosphere-derived cells expanded from percutaneous endomyocardial biopsy specimens, Circulation. 2007 Feb. 20; 115(7):896-908.

What is meant by “non-potent” cells according to the present invention is that such cells are incapable of improving a particular disease state. For instance, administration of “non-potent” cells in infarcted mice would lead to a change in ejection fraction similar to non-treated animals (ΔEF≤0%). Id.

What is meant by “borderline-potent” or “marginally-potent” cells according to the present invention is that such cells are somewhat capable of improving a particular disease state, and thus can be converted into “potent” activation-induced tissue-effector cells according to the method of the present invention. For instance, in the context of administration of “borderline-potent” or “marginally-potent” cells in infarcted mice would increase the left ventricular ejection fraction by at most 2% (i.e. ΔEF=0-2%). Id.

The term “exogenously” increasing the level of a transcription factor of interest in a cell has the plain and ordinary meaning in the field of cell therapy, namely, increasing the level or concentration of a transcription factor in a cell by the action of a molecular factor that originates from outside of the cell. Non-limiting examples include the use of a small molecule to interfere (by enhancement or inhibition) of one or more key pathway factors such as GSK3β, Axin1/2, β-catenin, and AKT1. Additionally, this could also be achieved by the introduction of transient or stable genetic material (through gene delivery mechanisms) that increases the availability of the transcription factor. Non-limiting examples include transfection by plasmids or other genetic material or the use of viral vectors. In contrast, merely selecting a cell, or a group of homogeneous population of cells, from a heterogeneous population of cells having various levels of cell potency, based on a preexisting or native high level of a particular transcription factor of interest would not be “exogenously” increasing the level of such a transcription factor of interest. For instance, what is meant by “exogenously” increasing the level of a transcription factor of interest, e.g. 0-catenin, of a non-potent or insufficiently potent cell is that that cell has a preexisting or native low level or concentration of the transcription factor of interest, such that that cell is already non-potent or insufficiently potent, before being subjected to the method according to the present invention for the production of activation-induced tissue-effector cells.

### BRIEF DESCRIPTION OF THE DRAWINGS

FIG. 1 shows a schematic representation of a method of making an activated specialized tissue-effector cell (ASTEC) according to one embodiment of the present invention.

FIG. 2 shows that activation of the canonical Wnt signaling pathway decreases the CD90 levels in CDCs.

FIGS. 3A and 3B show that activation of the canonical Wnt signaling pathway decreases the CD90 level in immortalized CDCs plated on a fibronectin (FN)-coated culture vessel, whereas no such effect is observed with immortalized CDCs plated on a CellBIND® (CB)-coated culture vessel.

FIG. 4 shows the chemical structure of CellBIND® surface treatment used to generate data shown in FIG. 3B.

FIG. 5 schematically shows the treatment of immortalized CDCs with Wnt3a to generate data shown in FIG. 3.

FIG. 6 shows that increasing passage number decreases the β-catenin level in CDCs.

FIG. 7 schematically shows the results shown in FIGS. 3-6.

FIG. 8 shows that immortalizing CDCs reduces their β-catenin levels.

FIGS. 9A and 9B show that BIO decreases the CD90 level and decreases the β-catenin level in primary CDCs, thereby showing that CD90 decrease is coupled with β-catenin increase in β-catenin.

FIG. 10 shows that the effect of BIO treatment lasts at least 24 hours after washout of BIO.

FIGS. 11A and 11B show that sphere formation upregulates β-catenin.

FIG. 12 shows that activation of β-catenin by BIO, or inactivation of β-catenin by JW67, improves cardiac recovery in a mouse model of acute myocardial infarction.

FIG. 13 shows that β-catenin levels are a positive indicator of cell potency as measured by the change in left ventricular ejection fraction (ΔEF) in a mouse model of acute myocardial infarction.

FIGS. 14A and 14B show that increasing the β-catenin level in the non-potent CDCs restores therapeutic potency in a mouse model of acute myocardial infarction.

FIGS. 15A and 15B show that increasing the β-catenin levels in the non-potent CDCs restores therapeutic potency in a mouse model of acute myocardial infarction.

FIGS. 16A and 16B show that increasing the β-catenin levels in the non-potent immortalized CDCs restores therapeutic potency in a mouse model of acute myocardial infarction.

FIGS. 17A and 17B show that increasing β-catenin levels in normal human dermal fibroblasts (NHDF) induces therapeutic potency in a mouse model of acute myocardial infarction.

FIG. 18A shows that kidney cortex cells express higher levels of baseline β-catenin than kidney medulla or proximal tubule cells.

FIG. 18B shows that BIO further increases β-catenin levels in kidney cortex cells.

FIG. 19 shows flow data of a primary CDC line that was immortalized using stable transduction of SV large T and small T antigens (T+t) paired with downregulation of the transcription factor mest, wherein three groups—a first group of primary (untransduced) CDCs, a second group of early passage (p. 8) immortalized CDCs, and a third group of a later passage (p. 17) CDCs—are compared to show changes over time.

FIG. 20 shows qPCR data of successful and sustained downregulation of the gene target mest, wherein three groups—a first group of primary (untransduced) CDCs, a second group of early passage (p. 7) immortalized CDCs, and a third group of a later passage (p. 12) CDCs—are compared to show changes over time.

FIG. 21 shows ELISA data of sustained upregulation of β-catenin levels across several passages of immortalized CDCs paired with downregulation of the transcription factor mest.

FIG. 22 shows that increasing the β-catenin level in immortalized CDCs paired with downregulation of the transcription factor mest increases therapeutic potency in a mouse model of acute myocardial infarction.

FIG. 23A and FIG. 23B shows flow data of surface expression of CD105 and CD90, and phase contrast images, respectively, of NHDFs, NHDFs with exogenously increased β-catenin level (NHDF^{βcat}) as an intermediate step, and NHDFs with exogenously increased β-catenin and GATA4 levels (NHDF^{βcat/gata4}).

FIG. 24 shows ELISA data of successful upregulation of β-catenin levels in NHDFs, NHDF^{βcat}, and NHDF^{βcat/gata4}.

FIG. 25 shows qPCR data of increased expression levels of GATA4, telomerase, Wnt-related genes in β-catenin signaling and regulation, and potency-related signals, in NHDFs, NHDF^{βcat}, and NHDF^{βcat/gata4}.

FIG. 26 shows Nanosight tracking data of size distribution of extracellular vesicles isolated from NHDFs, NHDF^{βcat}, and NHDF^{βCat/gata4}.

FIG. 27 shows qPCR data of a transition of the profile of extracellular vesicles isolated from NHDFs, NHDF^{βcat}, and NHDF^{βcat/gata4} towards a CDC-like profile including upregulation of miR-146a, miR-22, and miR-210, and downregulation of miR-199b.

FIG. 28 shows markedly improved survivorship in a mouse model of myocardial infarction treated with NHDF^{βcat} and NHDF^{βcat/gata4}

FIG. 29 shows that treatment with NHDF^{βcat/gata4} or extracellular vesicles derived from NHDF^{βcat/gata4} increases therapeutic potency functionally in a mouse model of myocardial infarction.

FIG. 30 shows that treatment with NHDF^{βcat/gata4} or extracellular vesicles derived from NHDF^{βcat/gata4} increases therapeutic potency structurally in a mouse model of myocardial infarction.

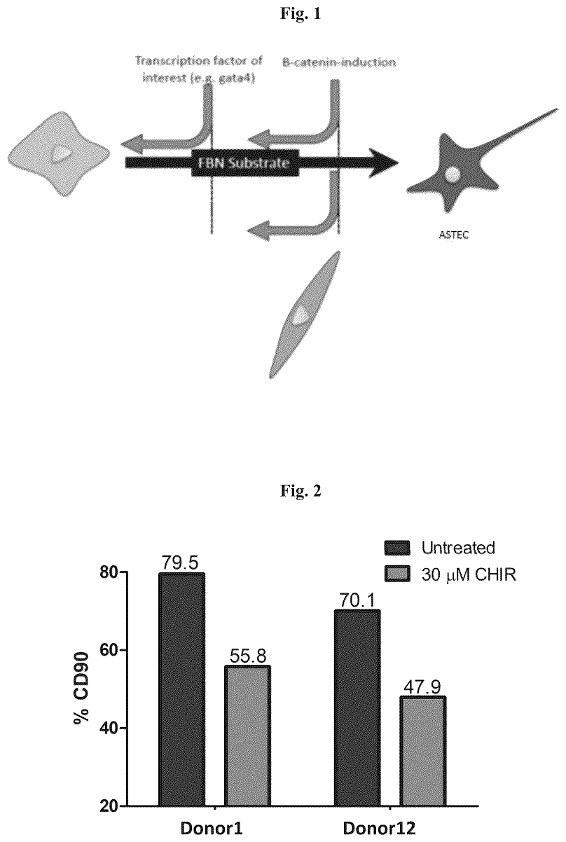

### DETAILED DESCRIPTION OF THE INVENTION

### A) Cardiospheres

Cardiospheres are undifferentiated cardiac cells that grow as self-adherent clusters as described in WO 2005/012510, and Messina et al., “Isolation and Expansion of Adult Cardiac Stem Cells from Human and Murine Heart,” Circulation Research, 95:911-921 (2004), the disclosures of which are herein incorporated by reference in their entirety.

Briefly, heart tissue can be collected from a patient during surgery or cardiac biopsy. The heart tissue can be harvested from the left ventricle, right ventricle, septum, left atrium, right atrium, crista terminalis, right ventricular endocardium, septal or ventricle wall, atrial appendages, or combinations thereof. A biopsy can be obtained, e.g., by using a percutaneous bioptome as described in, e.g., U.S. Patent Application Publication Nos. 2009/012422 and 2012/0039857, the disclosures of which are herein incorporated by reference in their entirety. The tissue can then be cultured directly, or alternatively, the heart tissue can be frozen, thawed, and then cultured. The tissue can be digested with protease enzymes such as collagenase, trypsin and the like. The heart tissue can be cultured as an explant such that cells including fibroblast-like cells and cardiosphere-forming cells grow out from the explant. In some instances, an explant is cultured on a culture vessel coated with one or more components of the extracellular matrix (e.g., fibronectin, laminin, collagen, elastin, or other extracellular matrix proteins). The tissue explant can be cultured for about 1, 2, 3, 4, or more weeks prior to collecting the cardiosphere-forming cells. A layer of fibroblast-like cells can grow from the explant onto which cardiosphere-forming cells appear. Cardiosphere-forming cells can appear as small, round, phase-bright cells under phase contrast microscopy. Cells surrounding the explant including cardiosphere-forming cells can be collected by manual methods or by enzymatic digestion. The collected cardiosphere-forming cells can be cultured under conditions to promote the formation of cardiospheres. In some aspects, the cells are cultured in cardiosphere-growth medium comprising buffered media, amino acids, nutrients, serum or serum replacement, growth factors including but not limited to EGF and bFGF, cytokines including but not limited to cardiotrophin, and other cardiosphere promoting factors such as but not limited to thrombin. Cardiosphere-forming cells can be plated at an appropriate density necessary for cardiosphere formation, such as about 20,000-100,000 cells/mL. The cells can be cultured on sterile dishes coated with poly-D-lysine, or other natural or synthetic molecules that hinder the cells from attaching to the surface of the dish. Cardiospheres can appear spontaneously about 2-7 days or more after cardiosphere-forming cells are plated.

### B) Cardiosphere-Derived Cells (CDCs)

CDCs are a population of cells generated by manipulating cardiospheres in the manner as described in, e.g., U.S. Patent Application Publication No. 2012/0315252, the disclosures of which are herein incorporated by reference in their entirety. For example, CDCs can be generated by plating cardiospheres on a solid surface which is coated with a substance which encourages adherence of cells to a solid surface of a culture vessel, e.g., fibronectin, a hydrogel, a polymer, laminin, serum, collagen, gelatin, or poly-D-lysine, and expanding same as an adherent monolayer culture. CDCs can be repeatedly passaged, e.g., passaged two times or more, according to standard cell culturing methods.

### C) Exosomes

Exosomes are vesicles formed via a specific intracellular pathway involving multivesicular bodies or endosomal-related regions of the plasma membrane of a cell. Exosomes can range in size from approximately 20-150 nm in diameter. In some cases, they have a characteristic buoyant density of approximately 1.1-1.2 g/mL, and a characteristic lipid composition. Their lipid membrane is typically rich in cholesterol and contains sphingomyelin, ceramide, lipid rafts and exposed phosphatidylserine. Exosomes express certain marker proteins, such as integrins and cell adhesion molecules, but generally lack markers of lysosomes, mitochondria, or caveolae. In some embodiments, the exosomes contain cell-derived components, such as but not limited to, proteins, DNA and RNA (e.g., microRNA and noncoding RNA). In some embodiments, exosomes can be obtained from cells obtained from a source that is allogeneic, autologous, xenogeneic, or syngeneic with respect to the recipient of the exosomes.

Certain types of RNA, e.g., microRNA (miRNA), are known to be carried by exosomes. miRNAs function as post-transcriptional regulators, often through binding to complementary sequences on target messenger RNA transcripts (mRNAs), thereby resulting in translational repression, target mRNA degradation and/or gene silencing. For example, as described in WO/2014/028493, miR146a exhibits over a 250-fold increased expression in CDCs, and miR210 is upregulated approximately 30-fold, as compared to the exosomes isolated from normal human dermal fibroblasts.

Exosomes derived from cardiospheres and CDCs are described in, e.g., WO/2014/028493, the disclosures of which are herein incorporated by reference in their entirety. Methods for preparing exosomes can include the steps of: culturing cardiospheres or CDCs in conditioned media, isolating the cells from the conditioned media, purifying the exosome by, e.g., sequential centrifugation, and optionally, clarifying the exosomes on a density gradient, e.g., sucrose density gradient. In some instances, the isolated and purified exosomes are essentially free of non-exosome components, such as components of cardiospheres or CDCs. Exosomes can be resuspended in a buffer such as a sterile PBS buffer containing 0.01-1% human serum albumin. The exosomes may be frozen and stored for future use.

Exosomes can be prepared using a commercial kit such as, but not limited to the ExoSpin™ Exosome Purification Kit, Invitrogen® Total Exosome Purification Kit, PureExo® Exosome Isolation Kit, and ExoCap™ Exosome Isolation kit. Methods for isolating exosome from stem cells are found in, e.g., Tan et al., Journal of Extracellular Vesicles, 2:22614 (2013); Ono et al., Sci Signal, 7(332):ra63 (2014) and U.S. Application Publication Nos. 2012/0093885 and 2014/0004601. Methods for isolating exosome from cardiosphere-derived cells are found in, e.g., Ibrahim et al., Exosomes as critical agents of cardiac regeneration triggered by cell therapy, Stem Cell Reports, 2014. Collected exosomes can be concentrated and/or purified using methods known in the art. Specific methodologies include ultracentrifugation, density gradient, HPLC, adherence to substrate based on affinity, or filtration based on size exclusion.

For example, differential ultracentrifugation has become a leading technique wherein secreted exosomes are isolated from the supernatants of cultured cells. This approach allows for separation of exosomes from nonmembranous particles, by exploiting their relatively low buoyant density. Size exclusion allows for their separation from biochemically similar, but biophysically different microvesicles, which possess larger diameters of up to 1,000 nm. Differences in flotation velocity further allows for separation of differentially sized exosomes. In general, exosome sizes will possess a diameter ranging from 30-200 nm, including sizes of 40-100 nm. Further purification may rely on specific properties of the particular exosomes of interest. This includes, e.g., use of immunoadsorption with a protein of interest to select specific vesicles with exoplasmic or outward orientations.

Among current methods, e.g., differential centrifugation, discontinuous density gradients, immunoaffinity, ultrafiltration and high performance liquid chromatography (HPLC), differential ultracentrifugation is the most commonly used for exosome isolation. This technique utilizes increasing centrifugal force from 2000×g to 10,000×g to separate the medium- and larger-sized particles and cell debris from the exosome pellet at 100,000×g. Centrifugation alone allows for significant separation/collection of exosomes from a conditioned medium, although it is insufficient to remove various protein aggregates, genetic materials, particulates from media and cell debris that are common contaminants. Enhanced specificity of exosome purification may deploy sequential centrifugation in combination with ultrafiltration, or equilibrium density gradient centrifugation in a sucrose density gradient, to provide for the greater purity of the exosome preparation (flotation density 1.1-1.2 g/mL) or application of a discrete sugar cushion in preparation.

Importantly, ultrafiltration can be used to purify exosomes without compromising their biological activity. Membranes with different pore sizes—such as 100 kDa molecular weight cut-off (MWCO) and gel filtration to eliminate smaller particles—have been used to avoid the use of a nonneutral pH or non-physiological salt concentration. Currently available tangential flow filtration (TFF) systems are scalable (to >10,000 L), allowing one to not only purify, but concentrate the exosome fractions, and such approaches are less time consuming than differential centrifugation. HPLC can also be used to purify exosomes to homogeneouslysized particles and preserve their biological activity as the preparation is maintained at a physiological pH and salt concentration.

Other chemical methods have exploit differential solubility of exosomes for precipitation techniques, addition to volume-excluding polymers (e.g., polyethylene glycols (PEGs)), possibly combined additional rounds of centrifugation or filtration. For example, a precipitation reagent, ExoQuick®, can be added to conditioned cell media to quickly and rapidly precipitate a population of exosomes, although re-suspension of pellets prepared via this technique may be difficult. Flow field-flow fractionation (FlFFF) is an elution-based technique that is used to separate and characterize macromolecules (e.g., proteins) and nano- to micro-sized particles (e.g., organelles and cells) and which has been successfully applied to fractionate exosomes from culture media.

Beyond these techniques relying on general biochemical and biophysical features, focused techniques may be applied to isolate specific exosomes of interest. This includes relying on antibody immunoaffinity to recognizing certain exosome-associated antigens. As described, exosomes further express the extracellular domain of membrane-bound receptors at the surface of the membrane. This presents a ripe opportunity for isolating and segregating exosomes in connections with their parental cellular origin, based on a shared antigenic profile. Conjugation to magnetic beads, chromatography matrices, plates or microfluidic devices allows isolating of specific exosome populations of interest as may be related to their production from a parent cell of interest or associated cellular regulatory state. Other affinity-capture methods use lectins which bind to specific saccharide residues on the exosome surface.

### C1) 10 KDa & 1000 KDa Method

CDC-EV (10 KDa or 1000 KDa) drug substance is obtained after filtering CDC conditioned medium (CM) containing EVs through a 10 KDa or 1000 KDa pore size filter, wherein the final product, composed of secreted EVs and concentrated CM, is formulated in PlasmaLyte A by diafiltration and stored frozen.

### C2) MSC-EVs

EVs originating from human bone marrow mesenchymal stem cells (MSC-EVs) are obtained after filtering MSC CM containing EVs through a 10 KDa pore size filter following a similar process as for CDC-EV production. MSC-EVs are a non-cellular, filter sterilized product obtained from human MSCs cultured under defined, serum-free conditions. The final product, composed of secreted EVs and concentrated CM, is formulated in PlasmaLyte A and stored frozen. The frozen final product is “ready to use” for direct subconjunctival injection after thawing.

### C3) Newt-EVs

EVs originating from newt Al cell line (Newt-EVs) are obtained after filtering Al cell line CM containing EVs through a 10 KDa pore size filter following a similar process as for CDC-EV production. Newt-EVs are a non-cellular, filter sterilized product obtained from newt Al cells cultured under defined, serum-free conditions. The final product, composed of secreted EVs and concentrated CM, is formulated in PlasmaLyte A and stored frozen. The frozen final product is ready to use for direct subconjunctival injection after thawing.

### D) Examples

The present invention is further described with reference to the following non-limiting examples.

### Example 1: CDC Culture

CDCs were prepared as described in U.S. Patent Application Publication No. 2012/0315252, the disclosures of which are herein incorporated by reference in their entirety.

In brief, heart biopsies were minced into small fragments and briefly digested with collagenase. Explants were then cultured on 20 mg/mL fibronectin-coated dishes. Stromal-like flat cells and phase-bright round cells grew out spontaneously from tissue fragments and reached confluency by 2-3 weeks. These cells were harvested using 0.25% trypsin and were cultured in suspension on 20 mg/mL poly-d-lysine to form self-aggregating cardiospheres. CDCs were obtained by plating and expanding the cardiospheres on a fibronectin-coated flask as an adherent monolayer culture. All cultures were maintained at 5% O₂, 5% CO₂ at 37° C., using IMDM basic medium supplemented with 10% FBS, 1% penicillin/streptomycin, and 0.1 mL 2-mercaptoethanol. CDCs were grown to 100% confluency on a fibronectin-coated flask to passage 5.

### Example 2: CDC Immortalization

CDCs were transduced with lentivirus containing genes for telomerase (hTert), simian virus serotype 40 large and small T antigens (SV40 T+t) or the cellular myelocytomatosis (c-Myc) gene. Briefly, 5×10⁴ CDCs (passage 2) were plated on a fibronectin-coated plate in a 24-well plate format. The cells were then treated with the aforementioned viruses at an MOI of 20 in complete media (10% FBS, pen/strep, 1-glut, and β-mercaptoethanol). The cells were transduced in the absence of a transduction reagent (namely polybrene) as previous observations have shown that it interferes with cell growth. The cells were passed as they became confluent (using complete media). At passage 5, the cells were passed from a T75 flask to a T25 flask, this time in the presence of the selection factor puromycin (5 pg/ml). When the cells were recovered and colonies began to form in the flask, the cells were passed and growth behavior was characterized well past the senescent stage of CDCs (passage 7-10).

CDC growth was monitored by counting the number of cells at every passage to derive the doubling rate of the cells. Briefly, the cells were passed 1:2 in a T175 flask format. When the cells were visibly confluent, the cells were trypsinized and were counted immediately before plating. The number of cells was compared to the number of the previous cell count. The cells were considered immortal if they continued to grow past passage 10.

### Example 3: Preparing Exosomes from Immortalized CDCs

Exosomes were derived from immortalized iCDCs in the same manner as described herein. Briefly, the cells were grown in T175 flasks. At confluence, the cells were washed twice with 30 ml of Iscove's Modified Dulbecco's Medium (IMDM). The cells were then conditioned in 32 ml of IMDM for a period of 15 days. At 15 days of conditioning, media was harvested and cleaned by spinning at 3000 g for 15 minutes. Conditioned media (CM) was aliquoted and stored at −80° C.

Exosomes size and concentration was measured using diffusion light scattering using a Malvern Nanosight instrument. Briefly, CM was diluted 1:10 in phosphatebuffered saline. To ensure accurate measurements, five (5)-60 sec videos were taken for each sample and batched together, and the data was pooled from all five videos of the same samples.

RNA from exosomes was isolated with a starting volume of 10 ml of CM using a Norgen Biotek Urine Exosome Isolation Kit. RNA was eluted in 50 μl of molecular grade water.

cDNA was made using High Capacity RNA to cDNA Kit by Life Technologies. Ct values were standardized using 18S primer and fold change was calculated based on empty vector control.

### Example 4: In Vitro Experiments

Referring to FIG. 2, CDCs from two donors (Donor1 and Donor12) were prepared as described herein. The attached CDCs were treated with 30 μM CHIR (Selleckchem, Cat. #S1263), a GSK30 inhibitor, or with no treatment for 72 hours. The cells were then collected, washed with 1% bovine serum albumin (BSA) in 1× phosphated-buffered saline (PBS), and stained with FITC Mouse Anti-Human CD90 (BD Biosciences, Cat. #555595). Flow cytometry was then conducted in BD FACSCanton™ II machine to measure CD90 expression in cells. As shown in FIG. 2, activation of the canonical Wnt signaling pathway decreased the CD90 levels in CDCs.

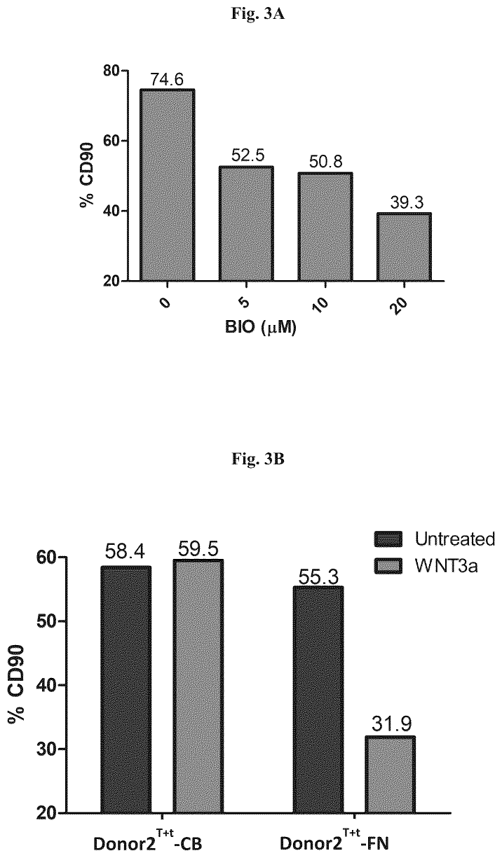

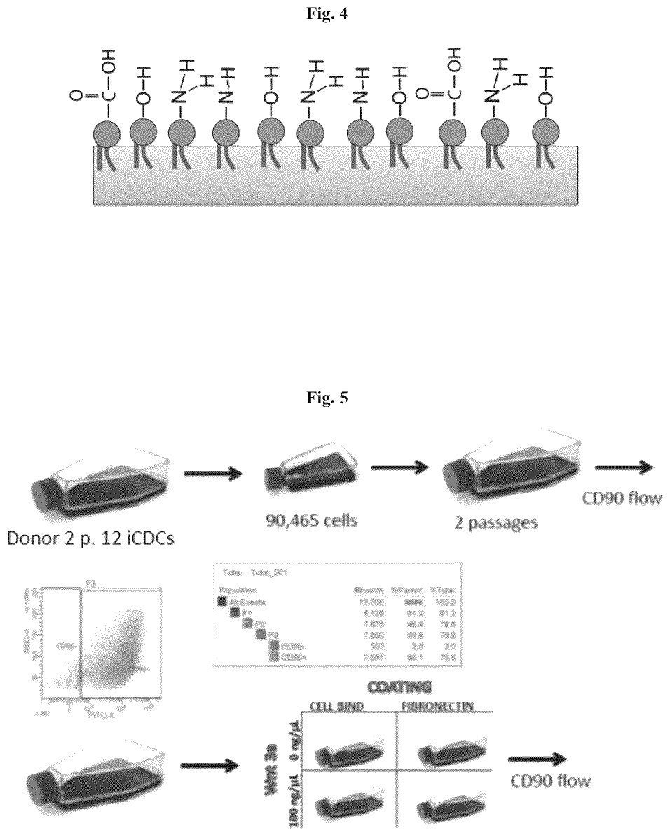

Referring to FIG. 3-5, CDCs from immortalized Donor2 were plated on either fibronectin (FN; VWR, Cat. #356009)-coated or CellBIND® (CB)-coated flasks. Attached cells were treated with Wnt3a or with PBS vehicle (ddH2O) for 72 hours. The cells were then analyzed using flow cytometry for CD90 expression with the same protocol as described herein. As shown in FIGS. 3A and 3B, activation of the canonical Wnt signaling pathway decreased the CD90 level in immortalized CDCs plated on a fibronectin (FN)-coated culture vessel, whereas no such effect is observed with immortalized CDCs plated on a CellBIND® (CB)-coated culture vessel.

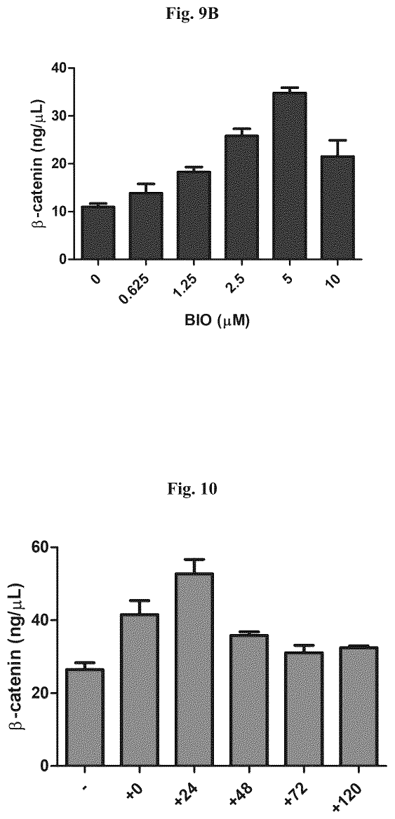

Referring to FIG. 9A, the flow data of CDCs from Donor5 shows decreasing CD90 levels with increasing BIO concentrations. Referring to FIG. 9B, CDCs from Donor 5 were treated with different concentrations of BIO (Sigma-Aldaich, Cat #B1685), a GSK30 inhibitor, for 72 hours. The cells were then collected and lysed with 1× lysis buffer supplied Total β-catenin ELISA (Affymetrix eBioscience InstantOne™ ELISA, Cat. #85-86141-11). Total β-catenin ELISA was performed as per manufacturer's instructions, except that 0.01 mg/mL final protein concentration was used in the assay. Concurrently, levels of β-catenin were increased with increasing BIO concentration.

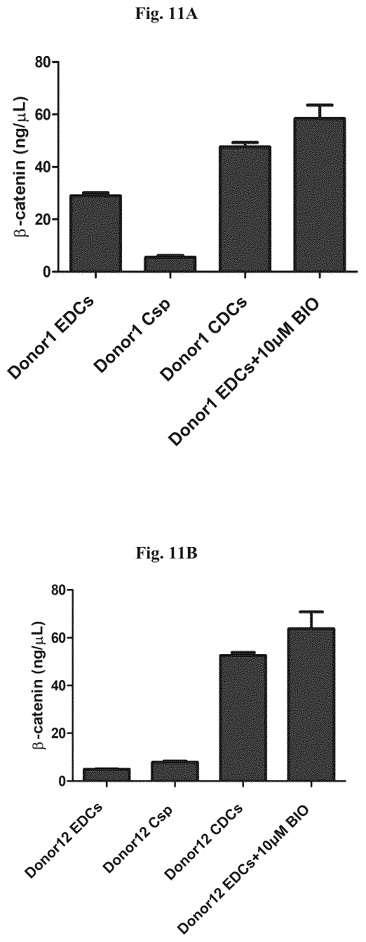

Referring to FIG. 10, the effect of BIO (as measured by sustained β-catenin activation per ELISA) was measured in CDCs from Donor10. Zero hour is immediately drug wash-off time point following a 72 hour of 5 μM BIO treatment period. The results show that peak activation is 24 hours after drug is washed out indicating the activity of internalized drug in the cells.

Referring to FIGS. 11A and 11B, sphere formation of explant-derived cells (EDCs) in UltraLow flask from two donors (Donor1 and Donor12) exhibited changing β-catenin levels (as measured by ELISA) throughout the 72-hour sphere-forming and resolution process. Treatment with BIO for 72 hours produced equal or better enhanced β-catenin expression.

### Example 5: Mouse Model of Acute Myocardial Infarction

To assess therapeutic efficacy in an established preclinical model, acute myocardial infarction (MI) was induced in immunodeficient mice in the same manner as described in, e.g., Ibrahim et al., Exosomes as critical agents of cardiac regeneration triggered by cell therapy, Stem Cell Reports, 2014. Briefly, 8-week old male severe combined immunodeficient (SCID) beige mice were anesthetized with isoflurane. Following surgical preparation, a 2 cm vertical incision was performed in the midclavicular line for a lateral thoracotomy. The left anterior descending was ligated using 7-0 silk. Animals then received intramyocardial injections of injected at two peri-infarct sites with 10⁵ cells (or phosphate buffered saline as a vehicle) in a total of 20 μl (10 μl/site). Echocardiography measurements were taken (to measure change left ventricular ejection fraction) at day 1 post and week 3 post infarction.

### Example 6: In Vivo Experiments

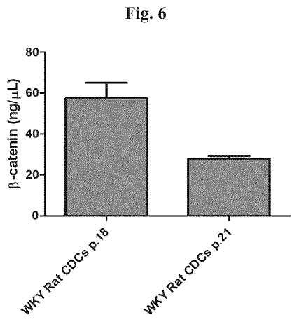

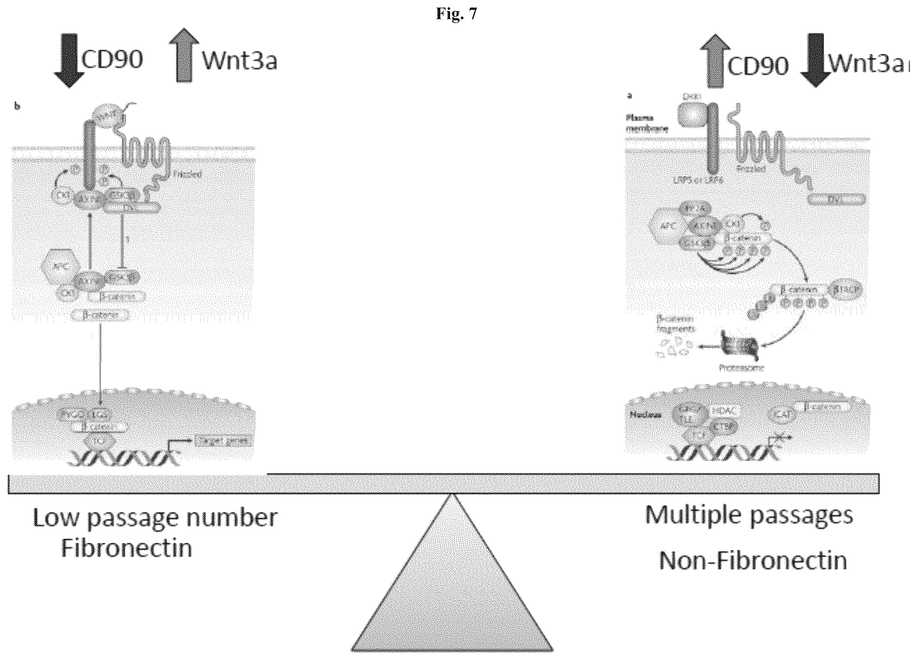

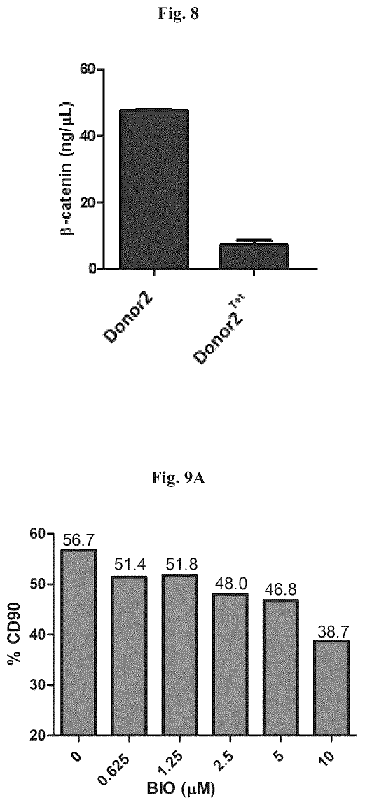

Referring to FIG. 6, CDCs derived from Wistar-Kyoto (WKY) rats were passaged and native β-catenin was measured at passages 18 and 21, wherein CDCs showed reduced levels of β-catenin (as measured by ELISA) as cell passage increases. This demonstrates that cell aging or senescence also leads to reduced potency.

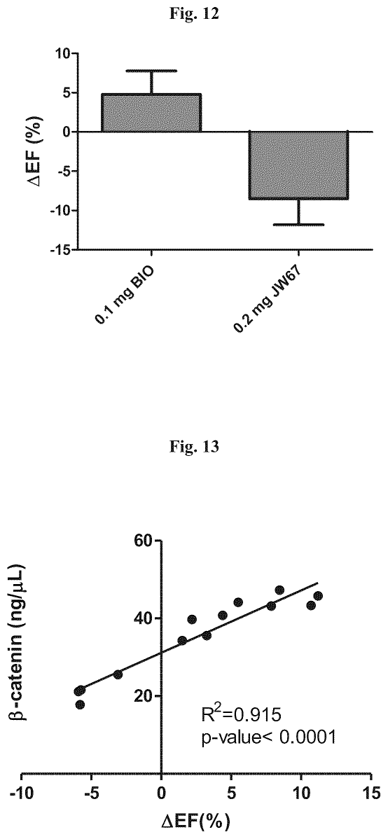

Referring to FIG. 12, mice with acute myocardial infarction were treated either by 0.1 mg of the β-catenin activating drug (BIO) or the β-catenin inhibitor (JW67). Cardiac ejection fraction change was measured three-week surgery, and the results demonstrate the role that β-catenin plays in cardiac recovery following infarction.

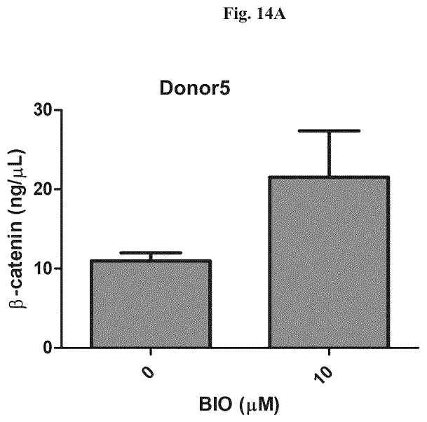

Referring to FIG. 13, a positive correlation between native β-catenin levels in CDCs and their potency (as measured by change in cardiac left ventricular ejection fraction [ΔEF]three weeks after CDC injection in the acute myocardial infarction mouse model; 10⁵ cells, intramyocardial injection at two peri-infarct sites) was observed.

Table 1 corresponds to FIG. 13, wherein each donor designation (e.g. Donor1) denotes particular CDCs which were derived from a particular individual heart in the same manner as described herein. Different lots from the same donor heart are denoted by an extra dash number designation (e.g. Donor6-1, 2, 3, 4 are all derived from the same donor heart but from different lots).

TABLE 1

| Donor | ΔEF (%) | β-catenin (ng/μl) |
|---|---|---|
| Donor1 | 11.2 | 45.8 |
| Donor2 | 8.5 | 47.3 |
| Donor3 | 10.7 | 43.3 |
| Donor4 | 4.4 | 40.8 |
| Donor5 | −5.8 | 17.8 |
| Donor6-1 | 7.9 | 43.2 |
| Donor6-2 | −5.7 | 21.6 |
| Donor6-3 | −5.9 | 21.2 |
| Donor6-4 | 1.5 | 34.3 |
| Donor7 | −3.1 | 25.5 |
| Donor8 | 3.3 | 35.6 |
| Donor9 | 5.5 | 44.1 |
| Donor10 | 2.2 | 39.7 |

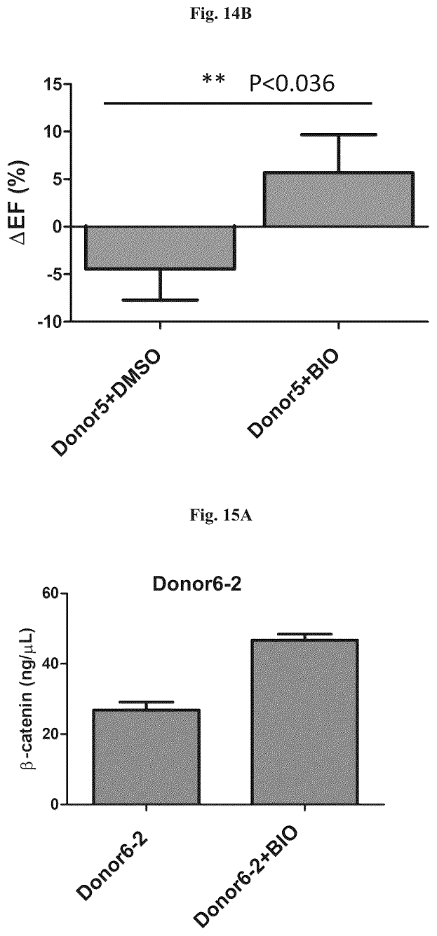

Referring to FIG. 14A, CDCs (10⁵ cells, intramyocardial injection at two peri-infarct sites) from Donor5 were treated with 0 μM or 10 μM BIO for 72 hours, wherein treatment with 10 μM BIO resulted in successful β-catenin restoration. Referring to FIG. 14B, the BIO-treated CDCs from Donor5 were injected into mice with acute myocardial infarction, and the results demonstrate potency of the BIO-treated cells as shown by increases in cardiac ejection fraction in the mouse MI model, with 95% confidence interval (Student's T-test).

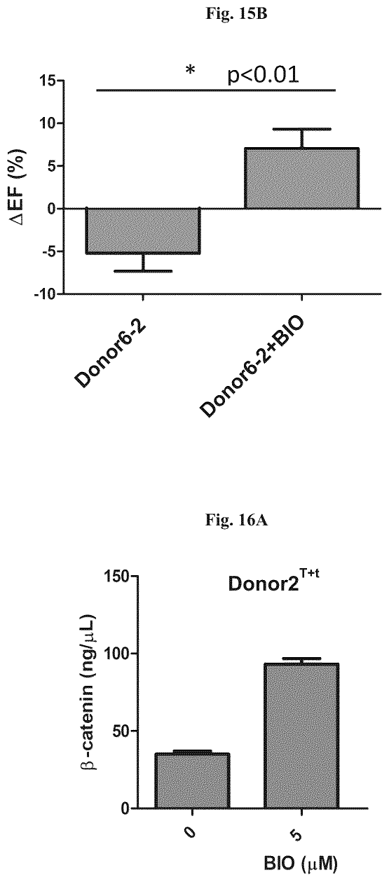

Referring to FIG. 15A, CDCs from Donor6-2 were treated with control vehicle or 5 μM BIO for 72 hours, wherein treatment with 5 μM BIO resulted in successful β-catenin restoration. Referring to FIG. 15B, the BIO-treated CDCs (10⁵ cells, intramyocardial injection at two peri-infarct sites) from Donor6-2 were injected into mice with acute myocardial infarction, and the results demonstrate potency of the BIO-treated cells as shown by increases in cardiac ejection fraction in the mouse model, with 95% confidence interval (Student's T-test).

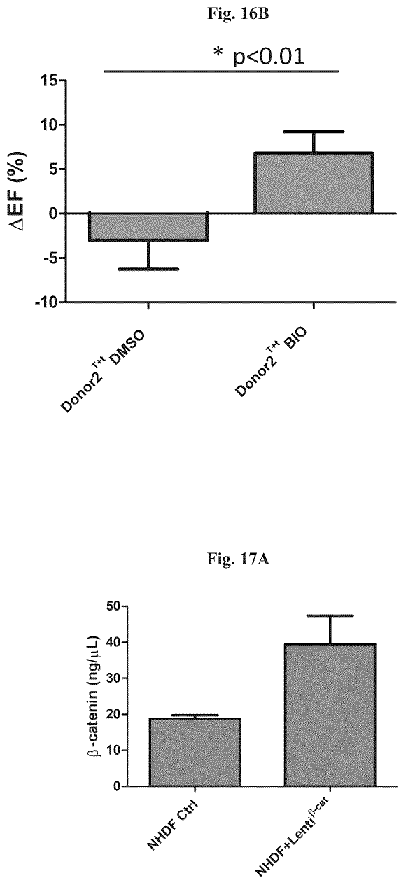

Referring to FIG. 16A, CDCs (10⁵ cells, intramyocardial injection at two peri-infarct sites) from Donor2 were immortalized by SV40 T+t lentivirus, and the immortalized CDCs from Donor2 were treated with 0 μM or 5 μM BIO for 72 hours, wherein treatment with 5 μM BIO resulted in successful β-catenin restoration. Referring to FIG. 16B, the BIO-treated immortalized CDCs from Donor2 were injected into mice with acute myocardial infarction, and the results demonstrate potency of the BIO-treated cells as shown by increases in cardiac ejection fraction in the mouse model, with 95% confidence interval (Student's T-test).

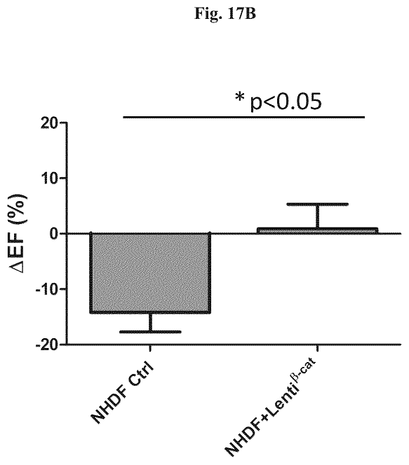

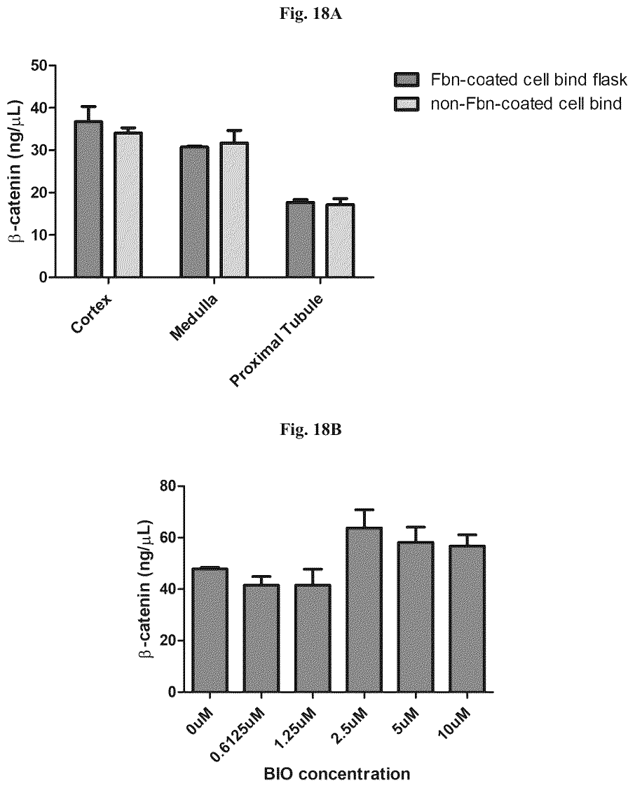

Referring to FIG. 17A, NHDFs were transduced with a lentivirus carrying β-catenin gene and resistance gene for puromycin (for selection) under the control of a constitutive expression promoter (NHDF+Lenti^{β-cat} cells), wherein the NHDF+Lenti^{β-cat} cells exhibited significantly higher levels of β-catenin compared to the control NHDFs which were transduced with empty vectors. Transduced cells were then selected for using puromycin antibiotic supplementation to complete media as the construct includes a puromycin resistance gene. Selection was done using 5 μg/ml of puromycin for 5 days. During the selection period media change was conducted each day for the first three days. Referring to FIG. 17B, NHDF+Lenti^{β-cat} cells (10⁵ cells, intramyocardial injection at two peri-infarct sites) were injected into mice with acute myocardial infarction, and the results demonstrate a greater increase in cardiac ejection fraction by NHDF+Lenti^{β-cat} cells compared to the control NHDFs which were transduced with empty vectors, with 95% confidence interval (Student's T-test). As such, the data presented in FIGS. 17A and 17B demonstrate that increasing β-catenin levels in non-potent NHDFs converts them into potent activation-induced tissue-effector cells.

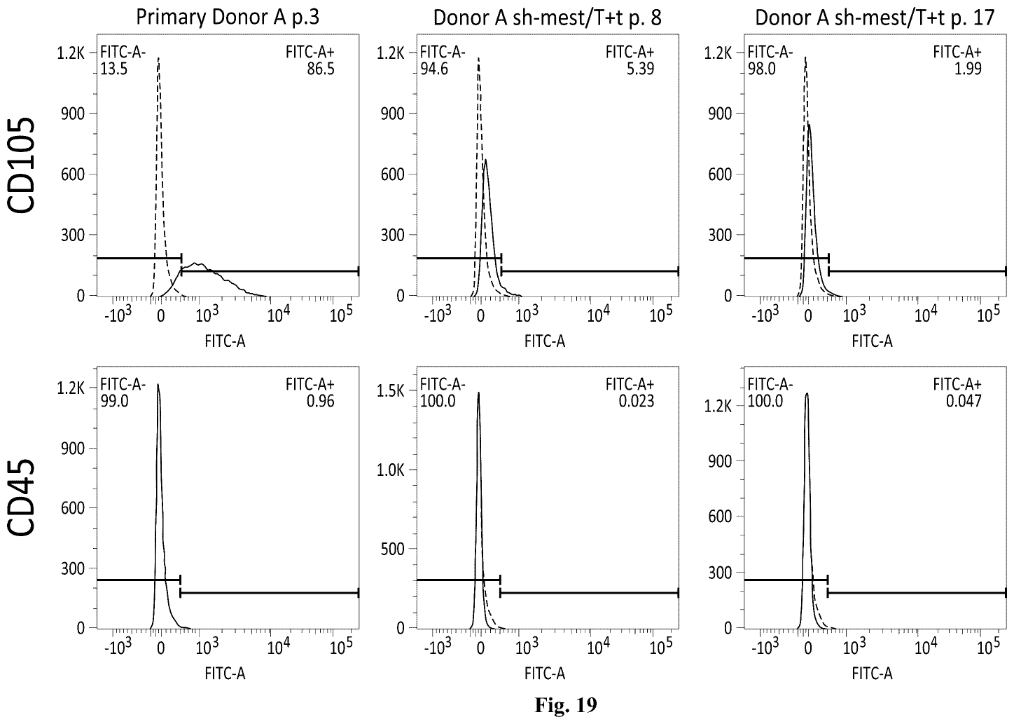

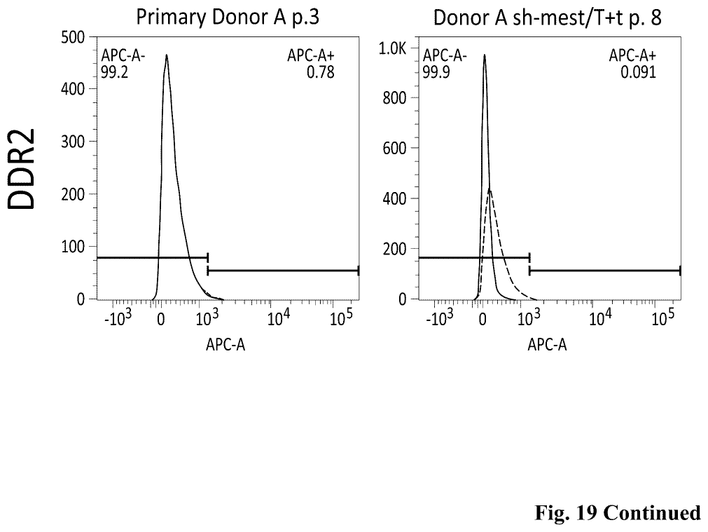

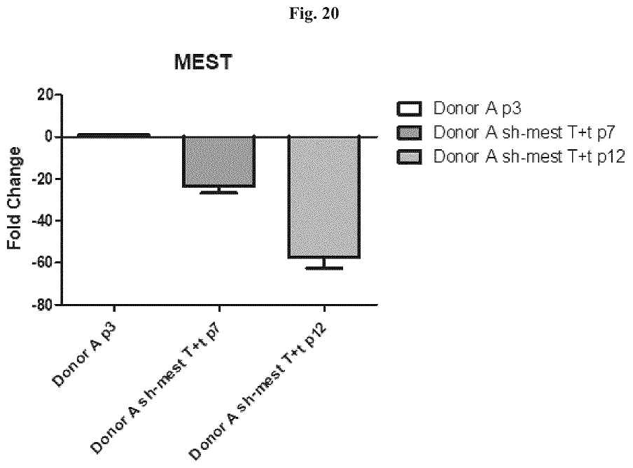

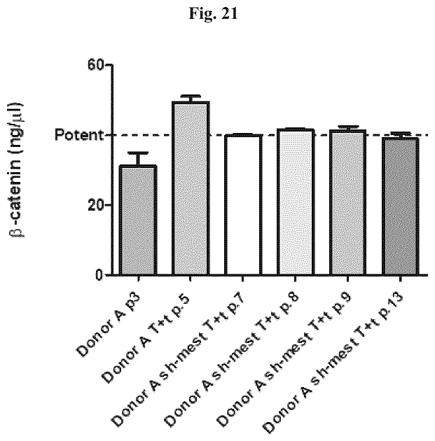

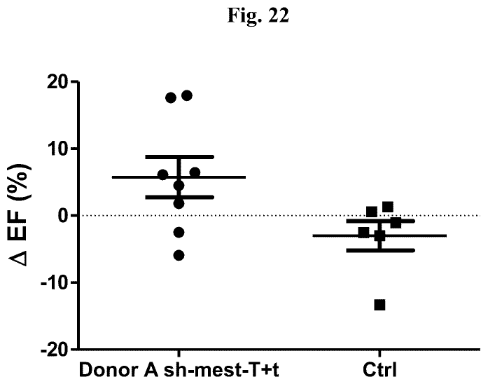

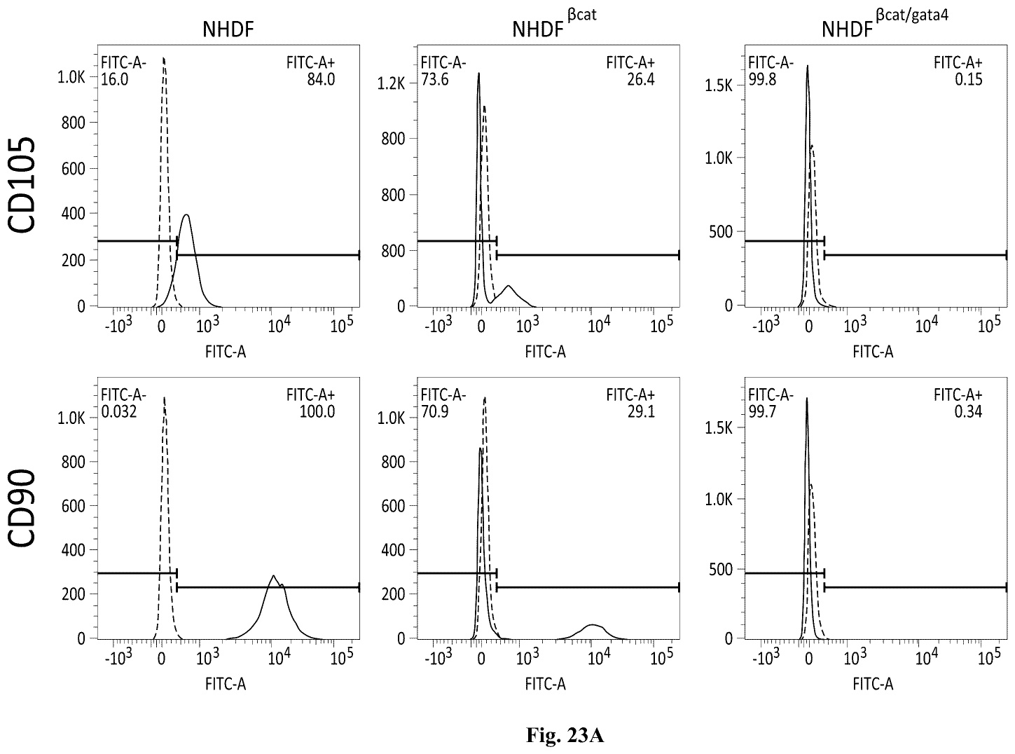

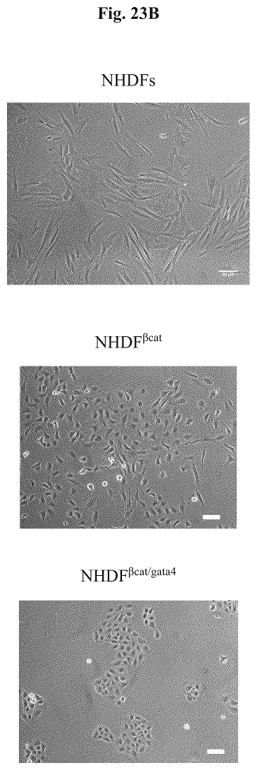

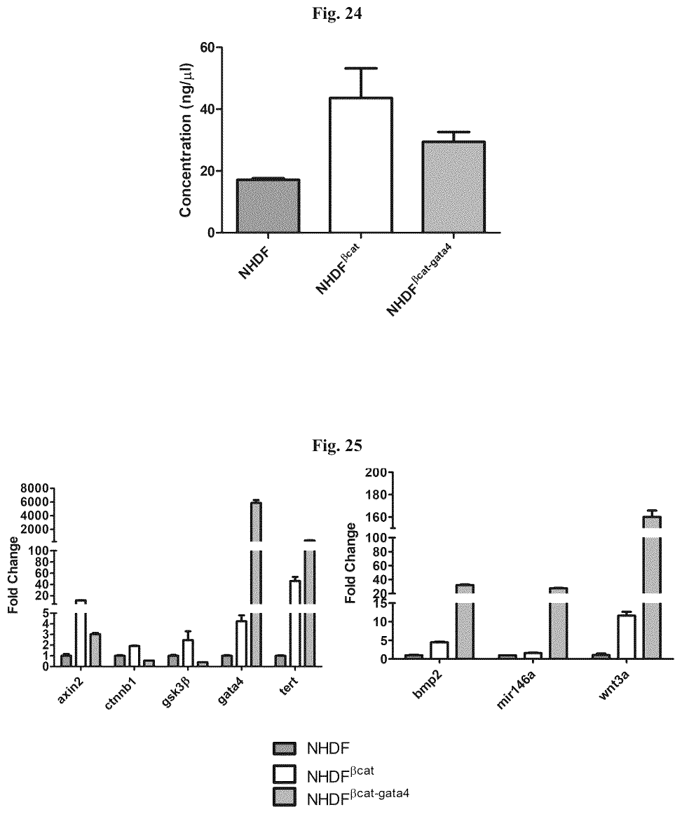

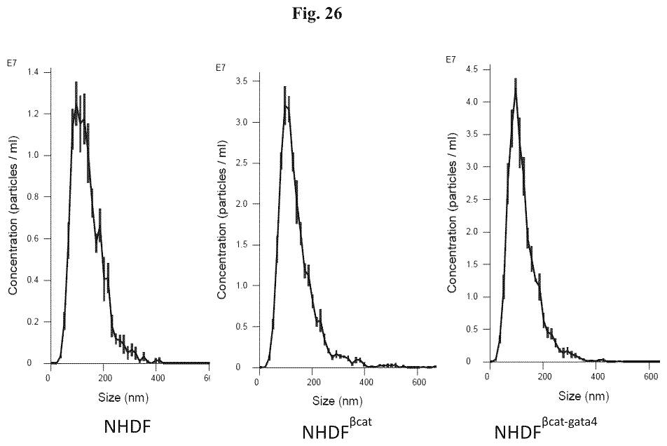

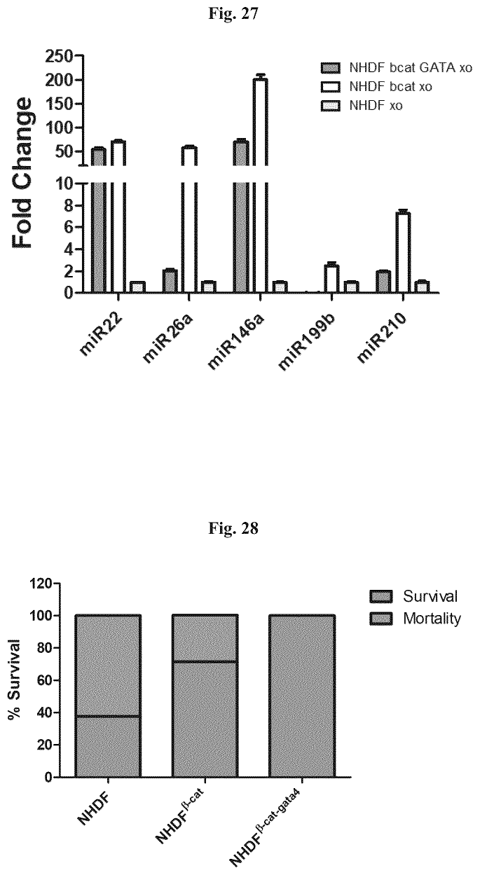

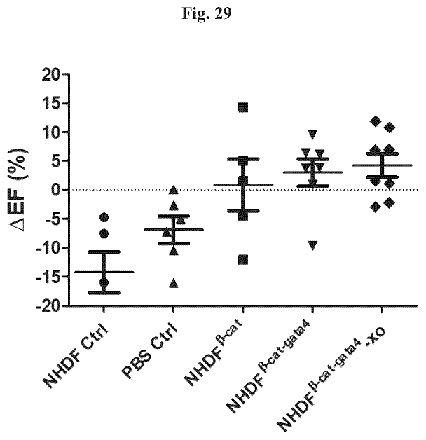

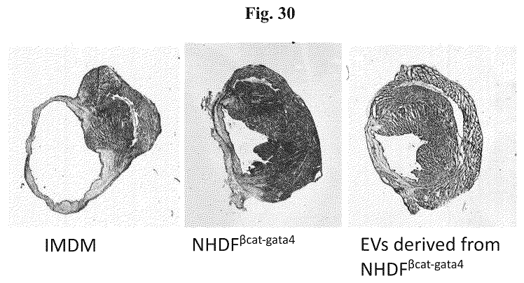

### Example 7: Experimental Methods for FIGS. 19-30

Cells and reagents. Endomyocardial biopsies from the right ventricular aspect of the interventricular septum were obtained from healthy hearts of decreased tissue donors. Cardiosphere-derived cells were derived as described previously. Briefly, heart biopsies were minced into small 1 mm² fragments and digested briefly with collagenase. Explants were then cultured on 20 μg/ml fibronectin (VWR)-coated flasks. Stromal-like, flat cells and phase-bright round cells grew spontaneously from the tissue fragments and reached confluence by 2-3 weeks. These cells were then harvested using 0.25% trypsin (GIBCO) and cultured in suspension on 20 μg/ml poly d-lysine (BD Biosciencs) to form self-aggregating cardiospheres. CDCs were obtained by seeding cardiospheres onto fibronectin-coated dishes and passaged. All cultures were maintained at 5% 02/C02 at 37° C., using IMDM basic media (GIBCO) supplemented with 10% FBS (Hyclone), 1% Gentamicin, and 0.1 ml 2-mercaptoethanol. Human heart biopsy specimens, from which CDCs were grown, were obtained under a protocol approved by the institutional review board from human-subject research.

CDC Exosome preparation and collection. Exosomes were harvested from primary CDCs at passage 5 or older passages from transduced cells. cells were grown to confluence at 5% 02/C02 at 37° C., washed three times with serum-free media and conditioned in serum-free media for 15 days. Conditioned media collected and filtered through 0.45 m filter to remove apoptotic bodies and cellular debris and frozen for later use at −80° C. To prepare exosomes for animal study, 2 ml PEG was added to 10 ml conditioned media on 4° C. rotator overnight to isolate the precipitated exosomes the next day.

Genetic modification/Transduction of cells. CDCs or NHDFs were plated on T25, and desired number of lentiviral particles were applied to flask with regular complete media when cells were attached to achieve MOI of 20. After 24 hr transduction, regular complete media was applied to flask to calm the cells down for another 24 hr. Selection media (complete media with desired antibody) was then added to flask for approximately one week to select transduced cells. Transduced cell RNA was collected and isolated once enough cells were obtained. qRT-qPCR was performed to verify the succeed of transduction.

RNA isolation and qRT-PCR. Total RNA was isolated using miRNeasy Mini Kit (Qiagen) for cells or Urine Exosome RNA Isolation Kit (Norgen Biotek Corp.) for exosomes. Reverse transcription was performed using High Capacity RNA to cDNA (Thermo Fisher Scientific) or Tagman® microRNA Reverse Transcription Kit (Applied Biosystems). Real-time PCR was performed using Taqman Fast Advanced Master Mix and the appropriate TaqMan® Gene Expression Assay (Thermo Fisher Scientific). The reaction was performed in QuantStudio™ 12K Flex Real-Time PCR system, and each reaction was performed in triplicated samples in target gene and housekeeping gene (HPRT1 for RNA and miR23a for microRNA) along with no template control.

Cell lysate and protein assay. 4×10⁵ cells were collected and pelleted at 1,000 rpm for 5 min at 4° C. Cell pellets were thoroughly mixed with 1× lysis buffer (Affymetrix eBioscience InstantOne ELISA kit) by vortexing and rotating for 10 min at room temperature. Protein lysate was isolated from the cell lysate mixture after centrifugation at 14,000 rpm for 15 min at 4° C. Protein concentration was determined by DC™ Protein Assay kit (Bio-Rad).

Drug treatment of cells. To increase β-catenin signaling in cells, 6-[[2-[[4-(2,4-Dichlorophenyl)-5-(5-methyl-1H-imidazol-2-yl)-2-pyrimidinyl]amino]ethyl]amino]-3-pyridinecarbonitrile (CHIR99021, Sigma-Aldaich) or 6-bromoindirubin-3′-oxime (BIO, Sigma-Aldaich), small molecules competitive inhibitor GSK3β, was used. Cells were harvested for desired experiments after treating with 30 μM CHIR or BIO serial dilution for 48 to 72 hours. On the other hand, Trispiro[3H-indole-3,2′-[1,3]dioxane-5′5″-[1,3]dioxane-2″,3′″-[3H]indole]-2,2′″(1H,1′″H)-dione(9C1) (JW67, Sigma-Aldaich) was applied for cell treatment in the same manner to decrease β-catenin signaling.

ELISA. Protein collection and protein concentration determination were as described above. Total β-catenin ELISA was performed according the protocol described with final sample concentration of 0.01 mg/ml and positive control of 0.1 mg/ml (ThermoFisher eBioscience InstantOne™ ELISA).

Flow cytometry. Cells were harvested and counted (2×10⁵ cells per condition). Cells were washed with 1% bovine serum albumin (BSA) in 1× Phosphated-buffered saline (PBS), and stained with the appropriate antibody (BD Pharmingen) for 1 hr at 4° C. The cells were then washed again and resuspended in 1% BSA in 1×PBS. BD Cytofix/Cytoperm™ kit was used for cell permeabilization before staining, such as LRP5/6 staining. The flow cytometry was performed in BD FACSCanto™ II machine.

Animal Study. All animal studies were conducted under the approval of Institutional Animal Care and Use Committee protocols. Acute myocardial infarction was applied to the three-month-old male severe combined immunodeficient (SCID)—beige mice as previously described. 1×10⁵ desired cells or 20 μl of 14 mM drug (BIO or JW67) or exosomes were injected to the SCID heart for rescuing.

Echocardiography. Echocardiography study was performed in the SCID beige in 24 hr (baseline) and 3 weeks after surgery using Vevo 3100 or 770 Imaging System (Visual Sonics) as described⁸. The average of the left ventricular ejection fraction was analyzed from multiple left ventricular end-diastolic and left ventricular end-systolic measurements.

Histology. Animals were sacrificed 3 weeks after MI. Hearts were harvested and a transverse cut was made slightly above the MI suture. The apical portion was then imbedded in optimum cutting temperature solution in a base mold/embedding ring block (Tissue Tek). Blocks were immediately frozen by submersion in cold 2-methylbutane. Hearts were sectioned at a thickness of 5 mM.

Masson's Trichrome Staining. Two slides containing a total of four sections per heart were stained using Masson's trichrome stain. In brief, sections were treated overnight in Bouin's solution. Slides were then rinsed for 10 min under running water and stained with Weigert's hematoxylin for 5 min. Slides were then rinsed and stained with scarlet-acid fuchsin for 5 min and rinsed again. Slides were then stained with phosphotungstic/phosphomolybdic, aniline blue, and 2% acetic acid for 5 min each. Slides were then rinsed, dried, and mounted using DPX mounting media.

1.-25. (canceled)

26. A method of making a therapeutic cell, the method comprising: (a) obtaining an immortalized cardiosphere-derived cell (CDC); and (b) treating the immortalized CDC with an exogenous agent to increase the level of beta-catenin in the immortalized CDC, thereby producing a therapeutically effective immortalized cell that improves cardiac function.

27. The method of claim 26, wherein the immortalized CDC is obtained by transforming a primary CDC.

28. The method of claim 26, wherein the exogenous agent is a GSK30 inhibitor.

29. The method of claim 27, wherein the said transforming comprises: overexpressing simian virus 40 large and small T antigens in a culture of CDCs; and selecting a CDC culture that can continue to double for at least 10 times.

30. The method of claim 27, wherein the said transforming comprises: overexpressing c-Myc in a culture of CDCs; and selecting a CDC culture that can continue to double for at least 10 times.

31. The method of claim 27, wherein the said transforming comprises: overexpressing hTert in a culture of CDCs; and selecting a CDC culture that can continue to double for at least 10 times.

32. The method of claim 26, wherein the immortalized CDC is cultured on a fibronectin (FN)-coated culture vessel.

33. The method of claim 26, further comprising selecting the therapeutically effective immortalized cell by CD90 flow cytometry, wherein the therapeutically effective immortalized cell has decreased expression of CD90 compared to an immortalized CDC.

34. The method of claim 26, further comprising: measuring the expression level of beta-catenin in the treated immortalized CDC; and selecting the therapeutically effective immortalized cell comprising beta-catenin at 34.3 ng/μL or greater.

35. The method of claim 27, further comprising: measuring the expression level of beta-catenin in the treated immortalized CDC; and selecting the therapeutically effective immortalized cell comprising beta-catenin at a level similar to a level of beta-catenin in the primary CDC.

36. The method of claim 26, wherein the immortalized CDC is treated for 72 hours with at least 5 μM 6-bromoindirubin-3′-oxime (BIO).

37. The method of claim 36, wherein the immortalized CDC is treated for 72 hours with at least 10 M BIO.

38. The method of claim 26, wherein the immortalized CDC is treated for 48 to 72 hours with 30 M 6-[[2-[[4-(2,4-Dichlorophenyl)-5-(5-methyl-1H-imidazol-2-yl)-2-pyrimidinyl]amino]ethyl]amino]-3-pyridinecarbonitrile (CHIR99021).

39. A composition comprising: a therapeutically effective cell; and a pharmaceutically acceptable excipient, wherein the therapeutically effective cell is an immortalized cardiosphere-derived cell (CDC) expressing at least 34.3 ng/μL beta-catenin and not expressing CD90.

40. The composition of claim 39, wherein the pharmaceutically acceptable excipient is sterile buffered saline.

41. The composition of claim 39, wherein the therapeutically effective cell is produced by: (a) obtaining a primary CDC; (b) immortalizing the primary CDC to make an immortal CDC; and (c) treating the immortalized CDC with a GSK30 inhibitor, thereby producing the therapeutically effective cell.

42. The composition of claim 41, wherein the therapeutically effective cell is selected by CD90 flow cytometry.

43. A method for improving heart function in a subject in need thereof, comprising administering a therapeutically effective cell to the subject, wherein the therapeutically effective cell is an immortalized cardiosphere-derived cell (CDC) expressing at least 34.3 ng/μL beta-catenin and not expressing CD90.

44. The method of claim 43, wherein the therapeutically effective cell is administered intravenously.

45. The method of claim 43, wherein the therapeutically effective cell is administered by intramyocardial injection.
